(19) United States
(12) Patent Application Publication
(10) Pub. No.: US 2009/0125022 A1
(43) Pub. Date: May 14, 2009

(54) TISSUE VISUALIZATION AND ABLATION SYSTEMS

(75) Inventors: Vahid SAADAT, Atherton, CA (US); Zachary J. MALCHANO, San Francisco, CA (US); David MILLER, Cupertino, CA (US); Ruey-Feng PEH, Mountain View, CA (US)

Correspondence Address: LEVINE BAGADE HAN LLP, 2483 EAST BAYSHORE ROAD, SUITE 100, PALO ALTO, CA, 94303, US

(73) Assignee: Voyage Medical, Inc., Campbell, CA (US)

(21) Appl. No.: 12/268,381

(22) Filed: Nov. 10, 2008

Related U.S. Application Data
(60) Provisional application No. 60/987,334, filed on Nov. 12, 2007.

(51) Int. Cl. A61B 18/14; A61B 1/00

(52) U.S. Cl. 606/41; 600/104

(57) ABSTRACT

Visualization and ablation system variations are described which utilize various tissue ablation arrangements. Such assemblies are configured to facilitate the application of energy delivery, such as RF ablation, to an underlying target tissue for treatment in a controlled manner while directly visualizing the tissue during the bipolar ablation process.

### CROSS-REFERENCE TO RELATED APPLICATIONS

This application claims the benefit of priority to U.S. Provisional Application No. 60/987,334, filed Nov. 12, 2007, which is incorporated herein by reference in its entirety.

### FIELD OF THE INVENTION

The present invention relates generally to medical devices used for accessing, visualizing, and/or treating regions of tissue within a body. More particularly, the present invention relates to methods and apparatus for the delivery of ablation energy, such as radio-frequency (RF) ablation, to an underlying target tissue for treatment in a controlled manner, while directly visualizing the tissue.

### BACKGROUND OF THE INVENTION

Conventional devices for visualizing interior regions of a body lumen are known. For example, ultrasound devices have been used to produce images from within a body in vivo. Ultrasound has been used both with and without contrast agents, which typically enhance ultrasound-derived images.

Other conventional methods have utilized catheters or probes having position sensors deployed within the body lumen, such as the interior of a cardiac chamber. These types of positional sensors are typically used to determine the movement of a cardiac tissue surface or the electrical activity within the cardiac tissue. When a sufficient number of points have been sampled by the sensors, a “map” of the cardiac tissue may be generated.

Another conventional device utilizes an inflatable balloon which is typically introduced intravascularly in a deflated state and then inflated against the tissue region to be examined. Imaging is typically accomplished by an optical fiber or other apparatus such as electronic chips for viewing the tissue through the membrane(s) of the inflated balloon. Moreover, the balloon must generally be inflated for imaging. Other conventional balloons utilize a cavity or depression formed at a distal end of the inflated balloon. This cavity or depression is pressed against the tissue to be examined and is flushed with a clear fluid to provide a clear pathway through the blood.

However, such imaging balloons have many inherent disadvantages. For instance, such balloons generally require that the balloon be inflated to a relatively large size which may undesirably displace surrounding tissue and interfere with fine positioning of the imaging system against the tissue. Moreover, the working area created by such inflatable balloons are generally cramped and limited in size. Furthermore, inflated balloons may be susceptible to pressure changes in the surrounding fluid. For example, if the environment surrounding the inflated balloon undergoes pressure changes, e.g., during systolic and diastolic pressure cycles in a beating heart, the constant pressure change may affect the inflated balloon volume and its positioning to produce unsteady or undesirable conditions for optimal tissue imaging. Additionally, imaging balloons are subject to producing poor or blurred tissue images if the balloon is not firmly pressed against the tissue surface because of intervening blood between the balloon and tissue.

Accordingly, these types of imaging modalities are generally unable to provide desirable images useful for sufficient diagnosis and therapy of the endoluminal structure, due in part to factors such as dynamic forces generated by the natural movement of the heart. Moreover, anatomic structures within the body can occlude or obstruct the image acquisition process. Also, the presence and movement of opaque bodily fluids such as blood generally make in vivo imaging of tissue regions within the heart difficult.

Other external imaging modalities are also conventionally utilized. For example, computed tomography (CT) and magnetic resonance imaging (MRI) are typical modalities which are widely used to obtain images of body lumens such as the interior chambers of the heart. However, such imaging modalities fail to provide real-time imaging for intra-operative therapeutic procedures. Fluoroscopic imaging, for instance, is widely used to identify anatomic landmarks within the heart and other regions of the body. However, fluoroscopy fails to provide an accurate image of the tissue quality or surface and also fails to provide for instrumentation for performing tissue manipulation or other therapeutic procedures upon the visualized tissue regions. In addition, fluoroscopy provides a shadow of the intervening tissue onto a plate or sensor when it may be desirable to view the intraluminal surface of the tissue to diagnose pathologies or to perform some form of therapy on it.

Thus, a tissue imaging system which is able to provide real-time in vivo images of tissue regions within body lumens such as the heart through opaque media such as blood and which also provide instruments for therapeutic procedures upon the visualized tissue are desirable.

### SUMMARY OF THE INVENTION

A tissue imaging and manipulation apparatus that may be utilized for procedures within a body lumen, such as the heart, in which visualization of the surrounding tissue is made difficult, if not impossible, by medium contained within the lumen such as blood, is described below. Generally, such a tissue imaging and manipulation apparatus comprises an optional delivery catheter or sheath through which a deployment catheter and imaging hood may be advanced for placement against or adjacent to the tissue to be imaged.

The deployment catheter may define a fluid delivery lumen therethrough as well as an imaging lumen within which an optical imaging fiber or assembly may be disposed for imaging tissue. When deployed, the imaging hood may be expanded into any number of shapes, e.g., cylindrical, conical as shown, semi-spherical, etc., provided that an open area or field is defined by the imaging hood. The open area is the area within which the tissue region of interest may be imaged. The imaging hood may also define an atraumatic contact lip or edge for placement or abutment against the tissue region of interest. Moreover, the distal end of the deployment catheter or separate manipulatable catheters may be articulated through various controlling mechanisms such as push-pull wires manually or via computer control

The deployment catheter may also be stabilized relative to the tissue surface through various methods. For instance, inflatable stabilizing balloons positioned along a length of the catheter may be utilized, or tissue engagement anchors may be passed through or along the deployment catheter for temporary engagement of the underlying tissue.

In operation, after the imaging hood has been deployed, fluid may be pumped at a positive pressure through the fluid delivery lumen until the fluid fills the open area completely and displaces any blood from within the open area. The fluid may comprise any biocompatible fluid, e.g., saline, water, plasma, Fluorinert™, etc., which is sufficiently transparent to allow for relatively undistorted visualization through the fluid. The fluid may be pumped continuously or intermittently to allow for image capture by an optional processor which may be in communication with the assembly.

In an exemplary variation for imaging tissue surfaces within a heart chamber containing blood, the tissue imaging and treatment system may generally comprise a catheter body having a lumen defined therethrough, a visualization element disposed adjacent the catheter body, the visualization element having a field of view, a transparent fluid source in fluid communication with the lumen, and a barrier or membrane extendable from the catheter body to localize, between the visualization element and the field of view, displacement of blood by transparent fluid that flows from the lumen, and an instrument translatable through the displaced blood for performing any number of treatments upon the tissue surface within the field of view. The imaging hood may be formed into any number of configurations and the imaging assembly may also be utilized with any number of therapeutic tools which may be deployed through the deployment catheter.

More particularly in certain variations, the tissue visualization system may comprise components including the imaging hood, where the hood may further include a membrane having a main aperture and additional optional openings disposed over the distal end of the hood. An introducer sheath or the deployment catheter upon which the imaging hood is disposed may further comprise a steerable segment made of multiple adjacent links which are pivotably connected to one another and which may be articulated within a single plane or multiple planes. The deployment catheter itself may be comprised of a multiple lumen extrusion, such as a four-lumen catheter extrusion, which is reinforced with braided stainless steel fibers to provide structural support. The proximal end of the catheter may be coupled to a handle for manipulation and articulation of the system.

To provide visualization, an imaging element such as a fiberscope or electronic imager such as a solid state camera, e.g., CCD or CMOS, may be mounted, e.g., on a shape memory wire, and positioned within or along the hood interior. A fluid reservoir and/or pump (e.g., syringe, pressurized intravenous bag, etc.) may be fluidly coupled to the proximal end of the catheter to hold the translucent fluid such as saline or contrast medium as well as for providing the pressure to inject the fluid into the imaging hood.

In treating tissue regions which are directly visualized, as described above, treatments utilizing electrical energy may be employed to ablate the underlying visualized tissue. Many ablative systems typically employ electrodes arranged in a monopolar configuration where a single electrode is positioned proximate to or directly against the tissue to be treated within the patient body and a return electrode is located external to the patient body. In other variations, biopolar configurations may be utilized.

In either case, in ablating the tissue via an electrode, any number of configurations may be utilized. For example, one variation of a hood may have a disc-shaped ablation electrode integrated upon the distal membrane and circumferentially positioned about the aperture. The disc-shaped electrode may be a solid or hollow conductive member (e.g., made of or coated with electrically conductive and biocompatible material such as gold, silver, platinum, Nitinol, etc.) electrically coupled via an insulated conductive wire or trace routed along or over the hood, e.g., along an inner edge of hood. The conductive wire or trace may be made from an electrically conductive material such as copper, stainless steel, Nitinol, silver, gold, platinum, etc. and insulated with a thin layer of non-conductive material such as latex or other biocompatible polymers.

In this and other variations described herein, the electrode may be utilized not only for tissue ablation treatment, but also for sensing or detecting any electrophysiological activity from the underlying tissue for mapping purposes. Additionally, the electrodes may also be used for pacing of cardiac tissue as well as for providing a form of confirmation of contact between the hood and cardiac tissue surfaces without the need of other imaging equipments such as fluoroscopy or ultrasound imaging.

Other variations may utilize an electrode fabricated from an optically transparent material which is biocompatible and electrically conductive, e.g., indium tin oxide, carbon nanotubes, etc. Yet other variations may utilize a mesh or grid of conductive wires which form a meshed electrode. Still other variations may utilize a separate wire electrode which may be shaped into various configurations, e.g., circular, positioned distal to the aperture.

This and other variations may additionally include a porous membrane where the aperture would normally be present such that the membrane defines a plurality of apertures or openings. The presence of a porous membrane may partially enclose the hood and slow the flow of the purging fluid from the interior of the hood. This low irrigation flow may still allow for cooling of the ablated tissue as well as facilitate conduction of electrical energy into the underlying tissue.

Other variations may further include one or more ridges or barriers defined over the distal membrane which extend just beyond the surface of the membrane. The ridges or barriers may extend in a radial pattern over the membrane and may number greater than or less than five ridges. The presence of such ridges may facilitate the uniform distribution of the purging saline fluid across the face of the hood which may in turn facilitate ablation and/or cooling of the underlying tissue. Additionally, the ridges or barriers may also prevent inadvertent slippage between the distal membrane the and the tissue surface by increasing friction and traction forces therebetween, particularly in areas where a thin layer of saline is able to weep across the surface due to non-uniform contact pressure distribution. Any of the other electrode configurations described herein, such as the disc-shaped electrode, may be utilized with this hood to facilitate ablation and cooling of the underlying tissue.

Yet another variation may utilize any of the electrode configurations described herein along with one or more additional apertures or openings defined about the main aperture. The presence of the additional openings increases the flow of the purging fluid from within the hood and may facilitate ablation and/or cooling of the underlying tissue. In yet another variation, the hood may be entirely closed by the presence of a solid disc-shaped electrode positioned upon the distal membrane of the hood. The size and shape of the resulting lesion upon the tissue surface may be modified by varying the size and shape of the electrode. In order to prevent the electrode from obstructing the view from the imaging element, an optically transparent and electrically conductive material may be used as previously described.

Optionally, a hood having any of the electrode configurations described herein may be coupled to a deployment catheter which has a flexible portion along the catheter shaft proximal to the hood. The flexible portion may be comprised of a bendable segment which may be passively or actively curved. Such a structure allows the hood to conform on the tissue surface regardless of the angle of approach which the hood takes relative to the tissue surface even when the hood approaches the tissue surface at an angle less than 90 degrees. Alternatively, the hood may itself comprise a flexible portion.

In yet another variation, the hood may comprise a flexible hood defining multiple apertures over the hood surface. A select number or all apertures may each have an electrode, such as a ring, disc-shaped, or any of the other electrode variations described herein, enclosing the apertures.

### BRIEF DESCRIPTION OF THE DRAWINGS

FIG. 1A shows a side view of one variation of a tissue imaging apparatus during deployment from a sheath or delivery catheter.

FIG. 1B shows the deployed tissue imaging apparatus of FIG. 1A having an optionally expandable hood or sheath attached to an imaging and/or diagnostic catheter.

FIG. 1C shows an end view of a deployed imaging apparatus.

FIGS. 2A and 2B show one example of a deployed tissue imager positioned against or adjacent to the tissue to be imaged and a flow of fluid, such as saline, displacing blood from within the expandable hood.

FIGS. 3A and 3B show examples of various visualization imagers which may be utilized within or along the imaging hood.

FIGS. 4A and 4B show perspective and end views, respectively, of an imaging hood having at least one layer of a transparent elastomeric membrane over the distal opening of the hood.

FIGS. 5A and 5B show perspective and end views, respectively, of an imaging hood which includes a membrane with an aperture defined therethrough and a plurality of additional openings defined over the membrane surrounding the aperture.

FIG. 6 illustrates an assembly view of one example of a visualization system configured with a grounding pad for ablation treatment.

FIG. 7 illustrates an assembly view of another example of a visualization system configured for visualized ablation while viewed upon a monitor.

FIGS. 8A to 8C respectively show perspective, side, and end views of one example of a tissue visualization and ablation system having a circularly-shaped electrode positioned about an aperture.

FIG. 8D shows a side view of the tissue visualization and ablation system of FIG. 8A positioned against a tissue surface to be ablated.

FIGS. 9A to 9C respectively show perspective, side, and end views of another example of a system having a transparent electrode placed about an aperture.

FIGS. 10A to 10C respectively show perspective, side, and end views of another example of a system having a meshed electrode positioned over an aperture.

FIG. 11A shows a perspective view of another example where the electrode is deployable as a ring structure separate from the hood and a hood having a meshed or porous aperture for decreasing an irrigation flow therethrough.

FIG. 11B shows the variation of FIG. 11A where the hood may comprise one or more ridges or barriers over the distal membrane to facilitate uniform cooling of the tissue undergoing ablation.

FIGS. 12A to 12E illustrate one variation for folding and/or retracting the hood of FIG. 11A into an outer sheath.

FIG. 13 shows a perspective view of another variation which utilizes a transparent electrode with a meshed or porous aperture for decreasing irrigation flow therethrough.

FIGS. 14A to 14C respectively show perspective, side, and end views of another example of a system having a circular electrode over the distal membrane with additional apertures defined therealong.

FIGS. 15A to 15C respectively show perspective, side, and end views of another example of a system having a disc-shaped electrode, which may be optionally transparent, positioned over the distal membrane.

FIG. 16 shows a perspective view of another variation having a circularly-shaped electrode, which may be optionally transparent, positioned over the distal membrane.

FIG. 17A shows a side view of another variation where one or more support struts serve as electrodes for ablating tissue via the one or more support struts.

FIG. 17B illustrates the device of FIG. 17A ablating tissue around the ostium of the superior right pulmonary vein in the left atrium.

FIGS. 18A and 18B show side views of another variation of a hood having a circularly-shaped electrode positioned, upon a deployment catheter having a flexible conforming portion to facilitate apposition of the hood upon a tissue surface.

FIGS. 19A and 19B show side views of another variation of the hood having a circularly-shaped electrode where the hood itself comprises a flexible conforming portion to facilitate apposition of the hood upon a tissue surface.

FIGS. 20A and 20B show side views of yet another variation of the hood having multiple apertures and multiple corresponding electrodes for positioning against a tissue surface.

FIGS. 21A to 21D illustrate variations of the hood positioned within a left atrium of a patient's heart treating the ostium around the pulmonary veins.

### DETAILED DESCRIPTION OF THE INVENTION

A tissue-imaging and manipulation apparatus described herein is able to provide real-time images in vivo of tissue regions within a body lumen such as a heart, which is filled with blood flowing dynamically therethrough and is also able to provide intravascular tools and instruments for performing various procedures upon the imaged tissue regions. Such an apparatus may be utilized for many procedures, e.g., facilitating transseptal access to the left atrium, cannulating the coronary sinus, diagnosis of valve regurgitation/stenosis, valvuloplasty, atrial appendage closure, arrhythmogenic focus ablation, among other procedures.

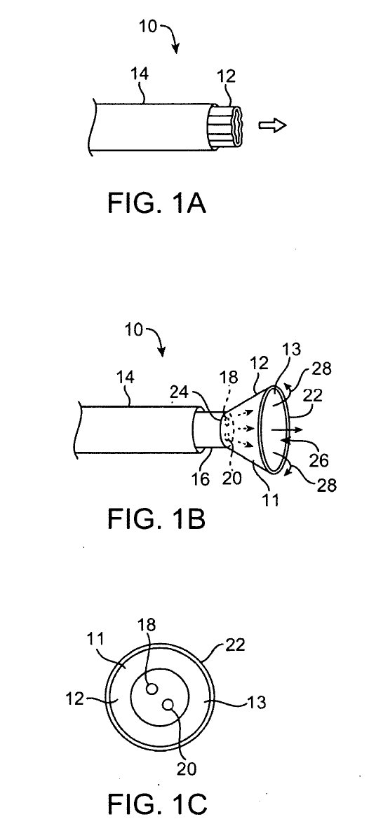

One variation of a tissue access and imaging apparatus is shown in the detail perspective views of FIGS. 1A to 1C. As shown in FIG. 1A, tissue imaging and manipulation assembly 10 may be delivered intravascularly through the patient's body in a low-profile configuration via a delivery catheter or sheath 14. In the case of treating tissue, it is generally desirable to enter or access the left atrium while minimizing trauma to the patient. To non-operatively effect such access, one conventional approach involves puncturing the intra-atrial septum from the right atrial chamber to the left atrial chamber in a procedure commonly called a transseptal procedure or septostomy. For procedures such as percutaneous valve repair and replacement, transseptal access to the left atrial chamber of the heart may allow for larger devices to be introduced into the venous system than can generally be introduced percutaneously into the arterial system.

When the imaging and manipulation assembly 10 is ready to be utilized for imaging tissue, imaging hood 12 may be advanced relative to catheter 14 and deployed from a distal opening of catheter 14, as shown by the arrow. Upon deployment, imaging hood 12 may be unconstrained to expand or open into a deployed imaging configuration, as shown in FIG. 1B. Imaging hood 12 may be fabricated from a variety of pliable or conformable biocompatible material including but not limited to, e.g., polymeric, plastic, or woven materials. One example of a woven material is Kevlar® (E. I. du Pont de Nemours, Wilmington, Del.), which is an aramid and which can be made into thin, e.g., less than 0.001 in., materials which maintain enough integrity for such applications described herein. Moreover, the imaging hood 12 may be fabricated from a translucent or opaque material and in a variety of different colors to optimize or attenuate any reflected lighting from surrounding fluids or structures, i.e., anatomical or mechanical structures or instruments. In either case, imaging hood 12 may be fabricated into a uniform structure or a scaffold-supported structure, in which case a scaffold made of a shape memory alloy, such as Nitinol, or a spring steel, or plastic, etc., may be fabricated and covered with the polymeric, plastic, or woven material. Hence, imaging hood 12 may comprise any of a wide variety of barriers or membrane structures, as may generally be used to localize displacement of blood or the like from a selected volume of a body lumen or heart chamber. In exemplary embodiments, a volume within an inner surface 13 of imaging hood 12 will be significantly less than a volume of the hood 12 between inner surface 13 and outer surface 11.

Imaging hood 12 may be attached at interface 24 to a deployment catheter 16 which may be translated independently of deployment catheter or sheath 14. Attachment of interface 24 may be accomplished through any number of conventional methods. Deployment catheter 16 may define a fluid delivery lumen 18 as well as an imaging lumen 20 within which an optical imaging fiber or assembly may be disposed for imaging tissue. When deployed, imaging hood 12 may expand into any number of shapes, e.g., cylindrical, conical as shown, semi-spherical, etc., provided that an open area or field 26 is defined by imaging hood 12. The open area 26 is the area within which the tissue region of interest may be imaged. Imaging hood 12 may also define an atraumatic contact lip or edge 22 for placement or abutment against the tissue region of interest. Moreover, the diameter of imaging hood 12 at its maximum fully deployed diameter, e.g., at contact lip or edge 22, is typically greater relative to a diameter of the deployment catheter 16 (although a diameter of contact lip or edge 22 may be made to have a smaller or equal diameter of deployment catheter 16). For instance, the contact edge diameter may range anywhere from 1 to 5 times (or even greater, as practicable) a diameter of deployment catheter 16. FIG. 1C shows an end view of the imaging hood 12 in its deployed configuration. Also shown are the contact lip or edge 22 and fluid delivery lumen 18 and imaging lumen 20.

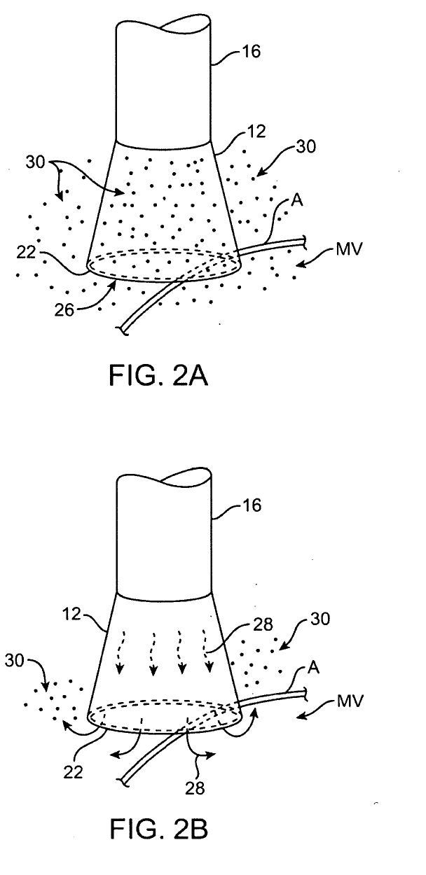

As seen in the example of FIGS. 2A and 2B, deployment catheter 16 may be manipulated to position deployed imaging hood 12 against or near the underlying tissue region of interest to be imaged, in this example a portion of annulus A of mitral valve MV within the left atrial chamber. As the surrounding blood 30 flows around imaging hood 12 and within open area 26 defined within imaging hood 12, as seen in FIG. 2A, the underlying annulus A is obstructed by the opaque blood 30 and is difficult to view through the imaging lumen 20. The translucent fluid 28, such as saline, may then be pumped through fluid delivery lumen 18, intermittently or continuously, until the blood 30 is at least partially, and preferably completely, displaced from within open area 26 by fluid 28, as shown in FIG. 2B.

Although contact edge 22 need not directly contact the underlying tissue, it is at least preferably brought into close proximity to the tissue such that the flow of clear fluid 28 from open area 26 may be maintained to inhibit significant backflow of blood 30 back into open area 26. Contact edge 22 may also be made of a soft elastomeric material such as certain soft grades of silicone or polyurethane, as typically known, to help contact edge 22 conform to an uneven or rough underlying anatomical tissue surface. Once the blood 30 has been displaced from imaging hood 12, an image may then be viewed of the underlying tissue through the clear fluid 30. This image may then be recorded or available for real-time viewing for performing a therapeutic procedure. The positive flow of fluid 28 may be maintained continuously to provide for clear viewing of the underlying tissue. Alternatively, the fluid 28 may be pumped temporarily or sporadically only until a clear view of the tissue is available to be imaged and recorded, at which point the fluid flow 28 may cease and blood 30 may be allowed to seep or flow back into imaging hood 12. This process may be repeated a number of times at the same tissue region or at multiple tissue regions.

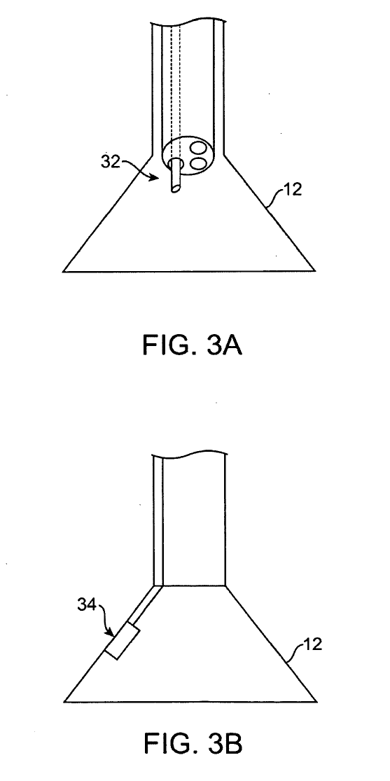

FIG. 3A shows a partial cross-sectional view of an example where one or more optical fiber bundles 32 may be positioned within the catheter and within imaging hood 12 to provide direct in-line imaging of the open area within hood 12. FIG. 3B shows another example where an imaging element 34 (e.g., CCD or CMOS electronic imager) may be placed along an interior surface of imaging hood 12 to provide imaging of the open area such that the imaging element 34 is off-axis relative to a longitudinal axis of the hood 12, as described in further detail below. The off-axis position of element 34 may provide for direct visualization and uninhibited access by instruments from the catheter to the underlying tissue during treatment.

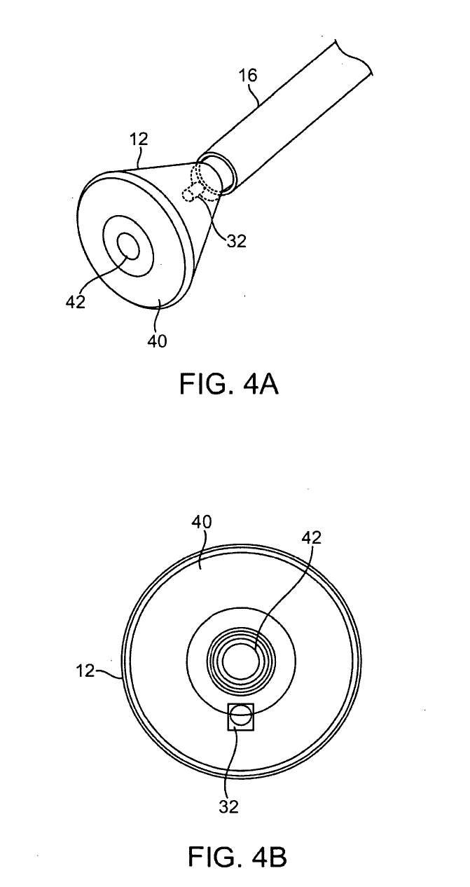

In utilizing the imaging hood 12 in any one of the procedures described herein, the hood 12 may have an open field which is uncovered and clear to provide direct tissue contact between the hood interior and the underlying tissue to effect any number of treatments upon the tissue, as described above. Yet in additional variations, imaging hood 12 may utilize other configurations. An additional variation of the imaging hood 12 is shown in the perspective and end views, respectively, of FIGS. 4A and 4B, where imaging hood 12 includes at least one layer of a transparent elastomeric membrane 40 over the distal opening of hood 12. An aperture 42 having a diameter which is less than a diameter of the outer lip of imaging hood 12 may be defined over the center of membrane 40 where a longitudinal axis of the hood intersects the membrane such that the interior of hood 12 remains open and in fluid communication with the environment external to hood 12. Furthermore, aperture 42 may be sized, e.g., between 1 to 2 mm or more in diameter and membrane 40 can be made from any number of transparent elastomers such as silicone, polyurethane, latex, etc. such that contacted tissue may also be visualized through membrane 40 as well as through aperture 42.

Aperture 42 may function generally as a restricting passageway to reduce the rate of fluid out-flow from the hood 12 when the interior of the hood 12 is infused with the clear fluid through which underlying tissue regions may be visualized. Aside from restricting out-flow of clear fluid from within hood 12, aperture 42 may also restrict external surrounding fluids from entering hood 12 too rapidly. The reduction in the rate of fluid out-flow from the hood and blood in-flow into the hood may improve visualization conditions as hood 12 may be more readily filled with transparent fluid rather than being filled by opaque blood which may obstruct direct visualization by the visualization instruments.

Moreover, aperture 42 may be aligned with catheter 16 such that any instruments (e.g., piercing instruments, guidewires, tissue engagers, etc.) that are advanced into the hood interior may directly access the underlying tissue uninhibited or unrestricted for treatment through aperture 42. In other variations wherein aperture 42 may not be aligned with catheter 16, instruments passed through catheter 16 may still access the underlying tissue by simply piercing through membrane 40.

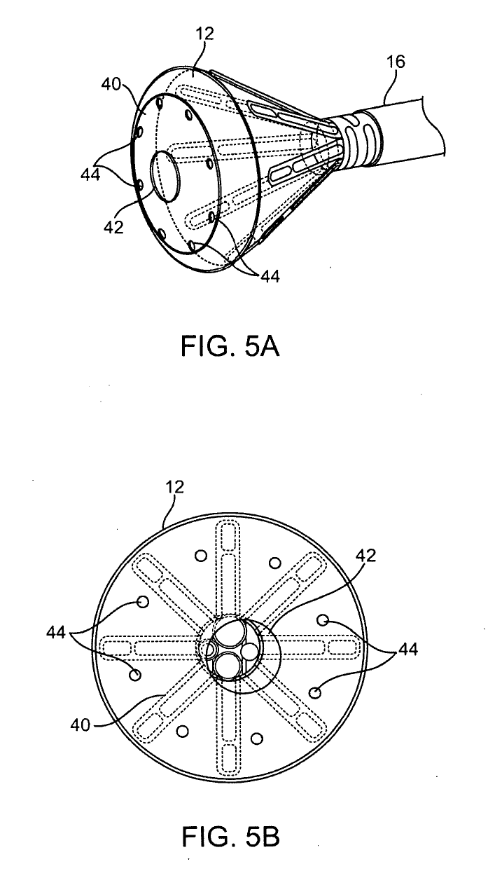

In an additional variation, FIGS. 5A and 5B show perspective and end views, respectively, of imaging hood 12 which includes membrane 40 with aperture 42 defined therethrough, as described above. This variation includes a plurality of additional openings 44 defined over membrane 40 surrounding aperture 42. Additional openings 44 may be uniformly sized, e.g., each less than 1 mm in diameter, to allow for the out-flow of the translucent fluid therethrough when in contact against the tissue surface. Moreover, although openings 44 are illustrated as uniform in size, the openings may be varied in size and their placement may also be non-uniform or random over membrane 40 rather than uniformly positioned about aperture 42 in FIG. 5B. Furthermore, there are eight openings 44 shown in the figures although fewer than eight or more than eight openings 44 may also be utilized over membrane 40.

Additional details of tissue imaging and manipulation systems and methods which may be utilized with apparatus and methods described herein are further described, for example, in U.S. patent application Ser. No. 11/259,498 filed Oct. 25, 2005 (U.S. Pat. Pub. No. 2006/0184048 A1); 11/763,399 filed Jun. 14, 2007 (U.S. Pat. Pub. No. 2007/0293724 A1); and also in 11/828,267 filed Jul. 25, 2007 (U.S. Pat. Pub. No. 2008/0033290 A1), and 11/775,837 filed Jul. 10, 2007 (U.S. Pat. Pub. No. 2008/0009747 A1) each of which is incorporated herein by reference in its entirety.

In treating tissue regions which are directly visualized, as described above, treatments utilizing electrical energy may be employed to ablate the underlying visualized tissue. Many ablative systems typically employ electrodes arranged in a monopolar configuration where a single electrode is positioned proximate to or directly against the tissue to be treated within the patient body and a return electrode is located external to the patient body. In other variations, biopolar configurations may be utilized.

In particular, such assemblies. apparatus, and methods may be utilized for treatment of various conditions, e.g., arrhythmias, through ablation under direct visualization. Details of examples for the treatment of arrhythmias under direct visualization which may be utilized with apparatus and methods described herein are described, for example, in U.S. patent application Ser. No. 11/775,819 filed Jul. 10, 2007 (U.S. Pat. Pub. No. 2008/0015569 A1), which is incorporated herein by reference in its entirety. Variations of the tissue imaging and manipulation apparatus may be configured to facilitate the application of bipolar energy delivery, such as radio-frequency (RF) ablation, to an underlying target tissue for treatment in a controlled manner while directly visualizing the tissue during the bipolar ablation process as well as confirming (visually and otherwise) appropriate treatment thereafter.

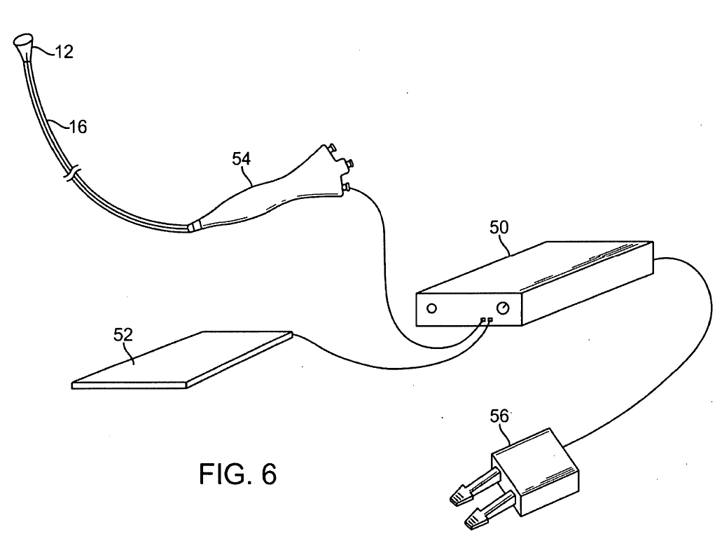

As illustrated in the assembly view of FIG. 6, hood 12 and deployment catheter 16 may be coupled to handle 54, through which the electrode may be coupled to the energy generator 50. The example illustrated shows a monopolar ablation configuration and thus includes grounding plate 52 also electrically coupled to generator 50. A separate actuation assembly 56, e.g., foot pedal, may also be electrically coupled to generator 50 to allow for actuation of the ablation energy. Upon filling the hood 12 with saline and obtaining a clear view of the tissue region of interest, the RF ablation energy generator 50 can be activated via actuation assembly 56 to initiate the flow of electrical currents to be transmitted from the generator 50 and through an ablation probe instrument, or through the purging fluid itself (e.g., saline) via an electrode to electrically charge the saline within the imaging hood 12, or through one or more electrodes positioned along or within the hood 12.

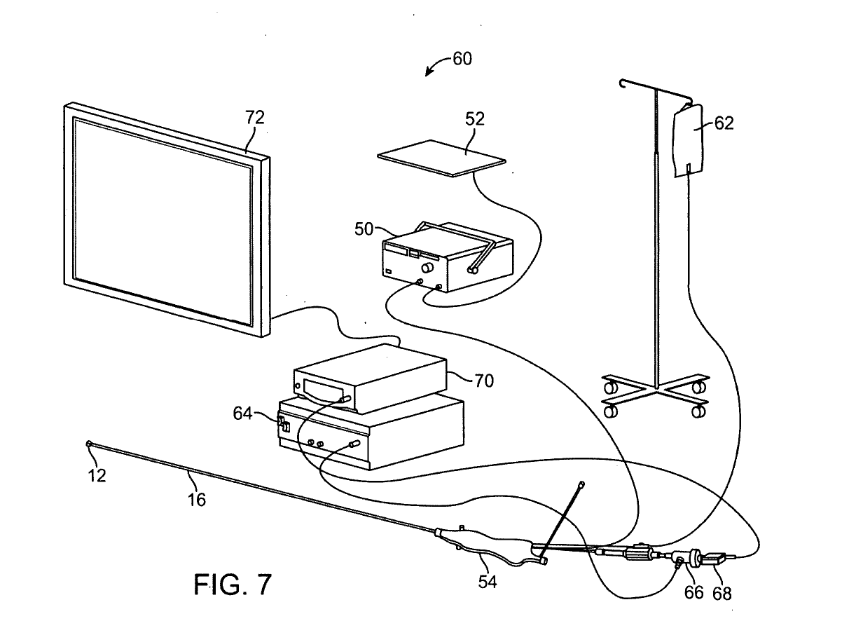

As the assembly allows for ablation of tissue directly visualized through hood 12, FIG. 7 illustrates an example of a system configured for enabling dual visualization and ablation. As shown in ablation assembly 60, hood 12 and deployment catheter 16 are coupled to handle 54, as previously described. Fluid reservoir 62, shown in this example as a saline-filled bag reservoir, may be attached through handle 54 to provide the clearing fluid and/or ablation medium. An optical imaging assembly 66 coupled to an imaging element positioned within or adjacent to hood 12 may extend proximally through handle 54 and be coupled to imaging processor assembly 64 for processing the images detected within hood 12. Assembly may also be coupled to a video receiving assembly 68 for receiving images from the optical imaging assembly 66. The video receiving assembly 68 may in turn be coupled to video processor assembly 70 which may process the detected images within hood 12 for display upon video display 72. Also shown are grounding plate 52 and ablation energy generator 50 which is coupled to ablation electrode within or proximate to hood 12, as previously described.

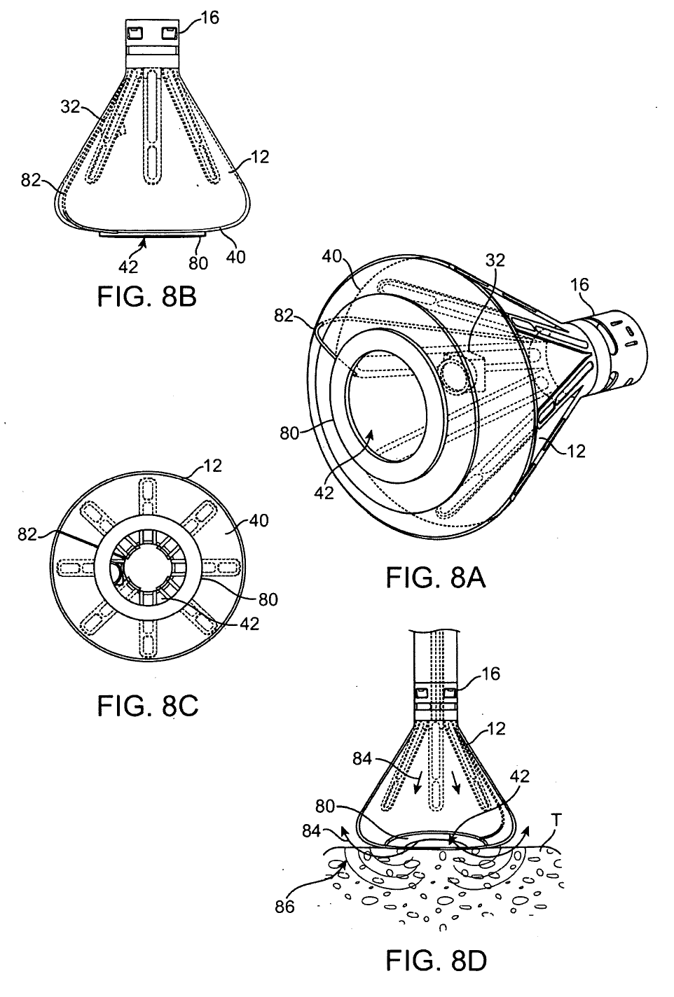

In ablating the tissue via an electrode, any number of configurations may be utilized. For example, FIGS. 8A to 8C show perspective, side, and end views, respectively, of one variation of a hood 12 having a disc-shaped ablation electrode 80 integrated upon the distal membrane 40 and circumferentially positioned about aperture 42. Disc-shaped electrode 80 may be a solid or hollow conductive member (e.g., made of or coated with electrically conductive and biocompatible material such as gold, silver, platinum, Nitinol, etc.) electrically coupled via an insulated conductive wire or trace 82 routed along or over hood 12, e.g., along an inner edge of hood 12. Conductive wire or trace 82 may be made from an electrically conductive material such as copper, stainless steel, Nitinol, silver, gold, platinum, etc. and insulated with a thin layer of non-conductive material such as latex or other biocompatible polymers. FIG. 8D illustrates a side view of hood 12 with electrode 80 placed into contact against a tissue region T to be treated. With the purging fluid 84 introduced into hood 12 and flowing out through the aperture, ablation energy 86 may be conducted from electrode 80 and into the underlying tissue region T.

In this and other variations described herein, the electrode may be utilized not only for tissue ablation treatment, but also for sensing or detecting any electrophysiological activity from the underlying tissue for mapping purposes. Additionally, the electrodes may also be used for pacing of cardiac tissue as well as for providing a form of confirmation of contact between the hood 12 and cardiac tissue surfaces without the need of other imaging equipments such as fluoroscopy or ultrasound imaging.

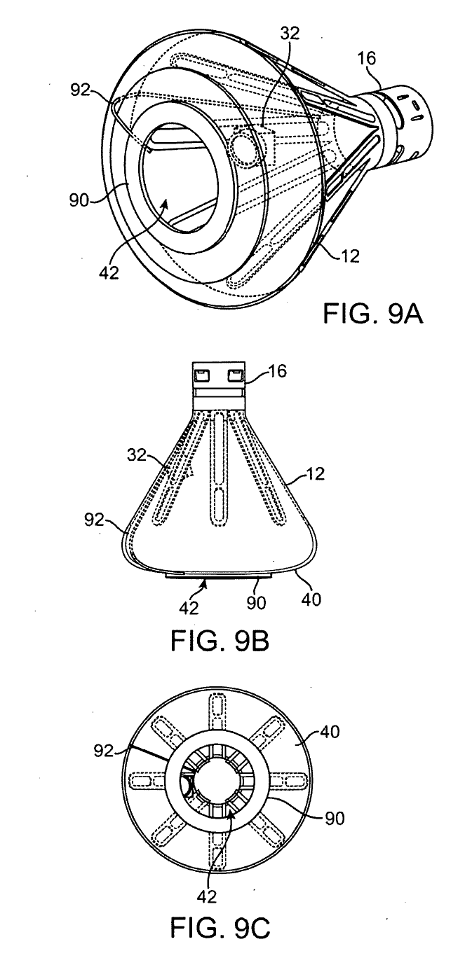

FIGS. 9A to 9C show perspective, side, and end views, respectively, of yet another variation in which hood 12 may have an electrode 90 positioned upon the distal membrane 40 of hood 12 where electrode 90 is fabricated from an optically transparent material which is biocompatible and electrically conductive, e.g., indium tin oxide, carbon nanotubes, etc. Transparent electrode 90 may be coupled to conductive wire or trace 92, as above. In utilizing transparent electrode 90, which may be generally fixed over distal membrane of hood 12, even the presence of electrode 90 may still allow for an unobstructed field of view of the underlying tissue by imager 32.

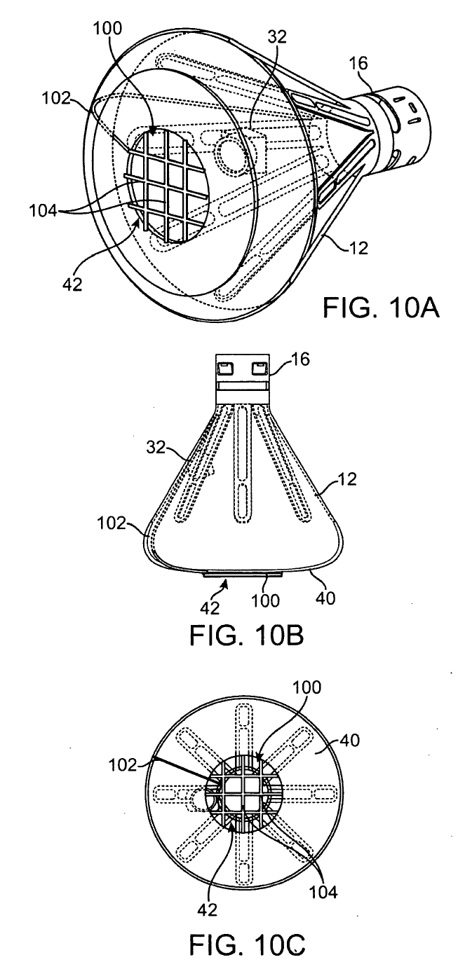

FIGS. 10A to 10C show perspective, side, and end views, respectively, of yet another variation where a mesh or grid of conductive wires 104 may form meshed electrode 100 coupled to conductive wire or trace 102, as previously described. The mesh or grid of wires 104 may be fitted over aperture 42 such that visualization of the underlying contacted tissue may still be performed. Moreover, the flow of visualization fluid through hood 12 may still flow through aperture 42 relatively unimpeded by meshed electrode 100 to facilitate ablation, clearing of blood, cooling of the tissue, etc. As above, the wires 104 of meshed electrode 100 may be constructed from any number of biocompatible materials which are electrically conductive (e.g., gold, silver, platinum, Nitinol, etc.).

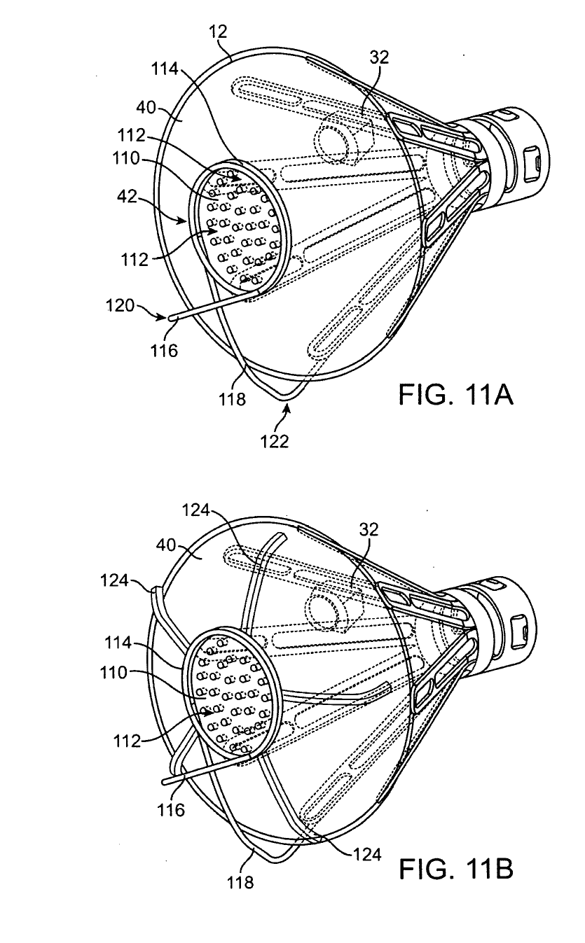

Another variation is shown in the perspective view of FIG. 11A, which shows hood 12 and a separate wire electrode 114, which may be shaped into various configurations, e.g., circular, positioned distal to the aperture 42. Electrode 114 may comprise a thermally insulated wire which is coated with a non-conductive material along its first and second conductive members 116, 118 which extend along an outer surface of hood 12 with respective conforming bends or curves 120, 122 to place the exposed electrode 114 distal to hood 12 adjacent to aperture 42. First and second conductive members 116, 118 may extend from electrode 114 at an angle relative to one another such that the members intersect one another to facilitate deployment and positioning of the electrode 114 when reconfigured from a low delivery profile to a deployment profile.

This variation may additionally include a porous membrane 110 where aperture 42 would normally be present such that the membrane 110 defines a plurality of apertures or openings 112. The presence of a porous membrane 110 may partially enclose the hood 12 and slow the flow of the purging fluid from the interior of hood 12. This low irrigation flow may still allow for cooling of the ablated tissue as well as facilitate conduction of electrical energy into the underlying tissue.

FIG. 11B illustrates a variation of FIG. 11A where hood 12 may further include one or more ridges or barriers 124 defined over distal membrane 40 which extend just beyond the surface of membrane 40. The ridges or barriers 124 may extend in a radial pattern over membrane 40 and may number greater than or less than five ridges 124, as shown. The presence of such ridges may facilitate the uniform distribution of the purging saline fluid across die face of hood 12 which may in turn facilitate ablation and/or cooling of the underlying tissue. Additionally, ridges or barriers 124 may also prevent inadvertent slippage between the distal membrane 40 and the tissue surface by increasing friction and traction forces therebetween, particularly in areas where a thin layer of saline is able to weep across the surface due to non-uniform contact pressure distribution. Any of the other electrode configurations described herein, such as the disc-shaped electrode, may be utilized with this hood to facilitate ablation and cooling of the underlying tissue.

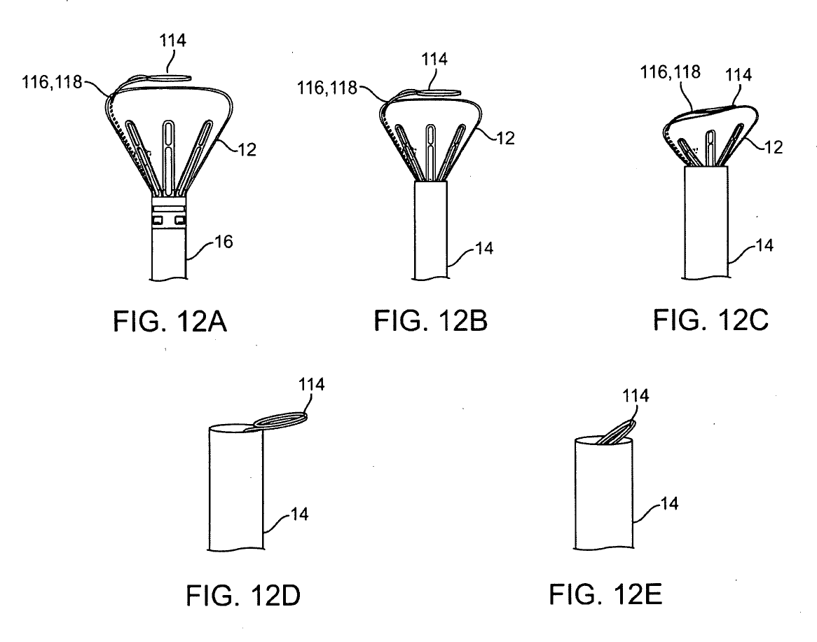

FIGS. 12A to 12E illustrate side views of one example for retracting the electrode and hood configuration described previously. FIG. 12A shows hood 12 and electrode 114 deployed distally of hood 12 via support members 116, 118. Hood 12 may be first retracted into outer sheath 14 while electrode 114, which may not be affixed to hood 12, may remain in its position, as shown in FIGS. 12B and 12C. Once hood 12 has been retracted within sheath 14, electrode 114 may then be pulled proximally into sheath 114 separately from hood 12, as illustrated in FIGS. 12D and 12E. Such a retraction method may enable both the hood 12 and a relatively larger electrode 114 to be deployed through a sheath 14 having a smaller relative diameter. The process may be reversed for deploying the electrode and hood within the body after delivery to a targeted tissue region of interest.

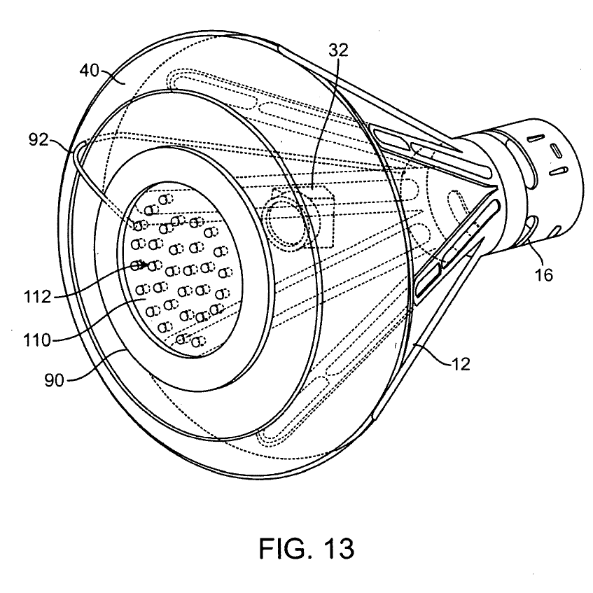

In yet another variation, an optically transparent (or at least partially transparent) circularly-shaped electrode may be employed to ensure views of the underlying tissue region are captured through the electrode 90, as previously described and as shown in the perspective view of FIG. 13. This variation may be utilized with the porous membrane 110 defining a plurality of pores or openings 112 to reduce the flow rate of the saline fluid from flowing externally of the hood 12. Such a mechanism may further serve to inhibit or prevent the entry of blood into hood 12 which can reduce the quality of visualization, increase hood purging times in obtaining clear visual images, and reduce the risk of blood coagulation.

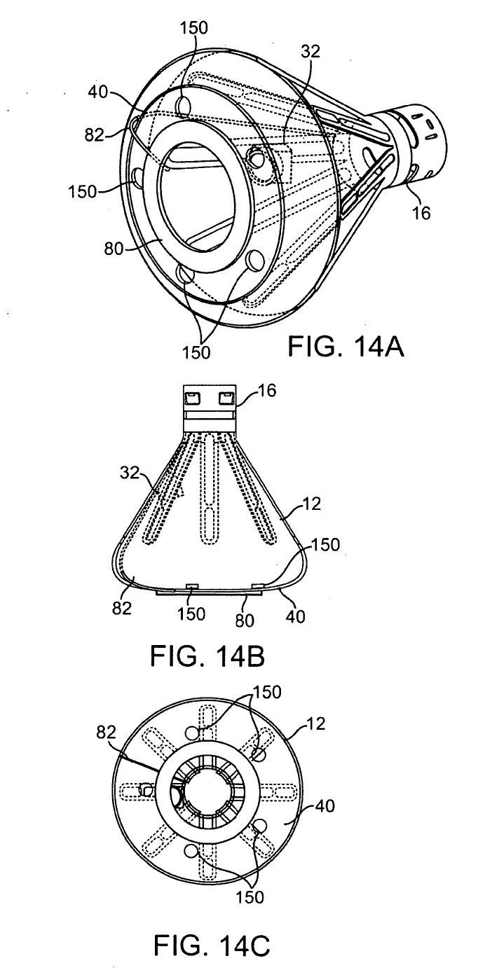

FIGS. 14A to 14C show yet another variation in the perspective, side, and end views, respectively, of hood 12 which may utilize any of the electrode configurations described herein, such as electrode 80, along with one or more additional apertures or openings 150 defined about the main aperture. The variation shown illustrates five additional apertures arranged in a circumferential (uniform or non-uniform) pattern about the main aperture although fewer than or more than five openings 150 may be utilized. The presence of the additional openings 150 increases the flow of the purging fluid from within hood 12 and may facilitate ablation and/or cooling of the underlying tissue.

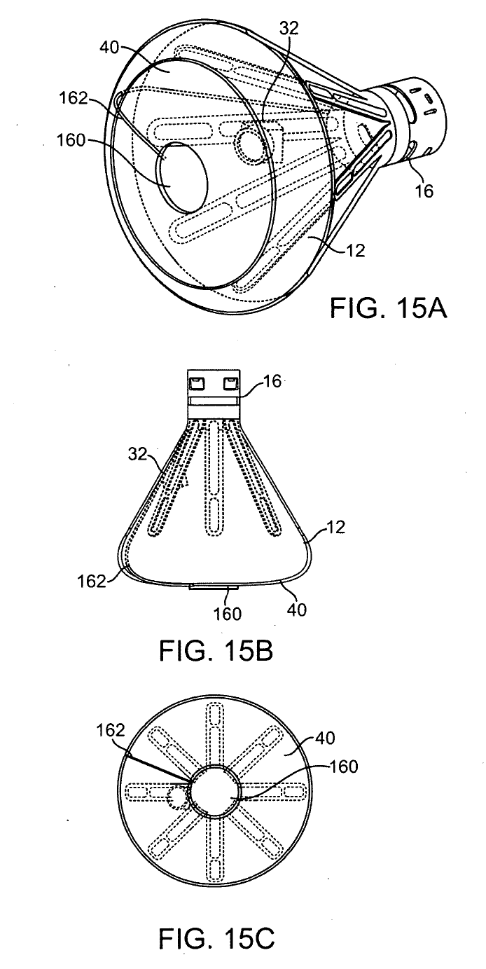

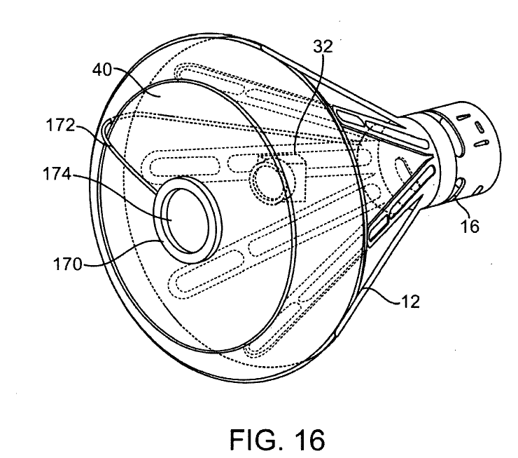

In yet another variation, FIGS. 15A to 15C show respective perspective, side, and end views of hood 12 which may be entirely closed by the presence of a solid disc-shaped electrode 160 electrically coupled via a conductive wire or trace 162, as previously described, positioned upon the distal membrane 40 of hood 12. The size and shape of the resulting lesion upon the tissue surface may be modified by varying the size and shape of the electrode 160. In order to prevent the electrode 160 from obstructing the view from imaging element 32, an optically transparent and electrically conductive material may be used as previously described. FIG. 16 shows a perspective view of a similar variation where a circularly shaped transparent electrode 170 coupled via conductive wire or trace 172 may be positioned upon distal membrane 40 which has a closed membrane 174 where the main aperture would normally be defined.

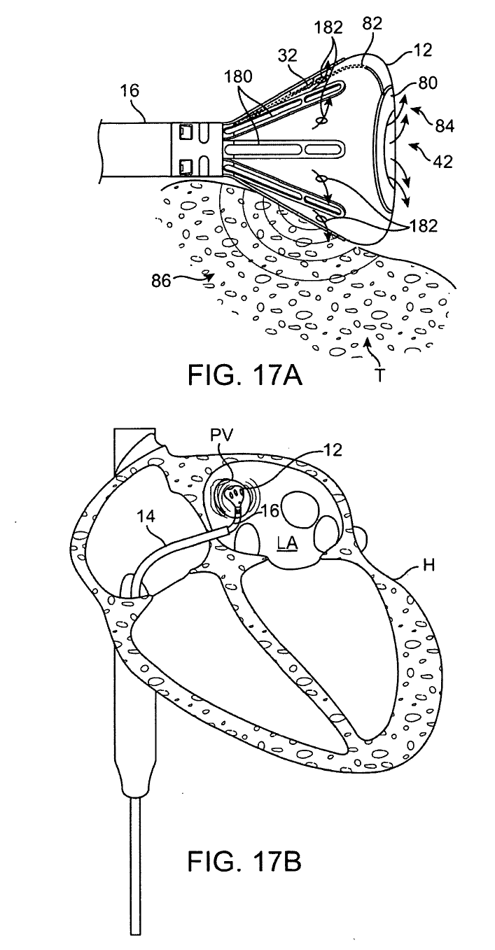

FIG. 17A shows a side view of another variation where one or more electrically conductive support struts 180 may be function as a return electrode to conduct electricity from electrode 80. These electrode support struts 180 may be positioned along hood 12 such that they are exposed exteriorly along an outer surface of hood 12. The conductive fluid 84 flowing through hood 12 may flow out of the main aperture 42 and out of a number of smaller apertures 182 defined along the side walls of hood 12 to flow around the electrode struts 180 such that energy is conducted between the struts 180. Because of the positioning of the struts along an exterior surface of hood 12, the hood outer surface may be utilized to contact and ablate underlying tissue. The flow of ablation energy 86 through the electrically charged fluid 84 between the struts 180 may result in the formation of lesions on the tissue region under the base of the hood 12 as well as along the side surfaces of the hood 12. The smaller apertures 182 may be defined between adjacent support struts 180 along the sides of the hood 12 to facilitate the uniform distribution of saline fluid over the ablated tissue.

Such a variation can be utilized to ablate tissue regions that are generally difficult to access by the hood 12 due to the relatively tight bend radius potentially needed to access the region or due to space constraints. FIG. 17B illustrates a cross-sectional side view of a hood 12 having the electrode configuration along the side surfaces advanced into contact against the tissue region, e.g., around the ostium of the right superior pulmonary vein PV, inside the left atrium LA of the heart H without having to conform into a tight bend radius. The imager 32 within the hood 12 can be manipulated to visualize the tissue region and potentially around the sides of the imaging hood 12 during the ablation process.

Additional examples of this variation are further described in detail in U.S. patent application Ser. No. 12/209,057 filed Sep. 11, 2008, which is incorporated herein by reference in its entirety.

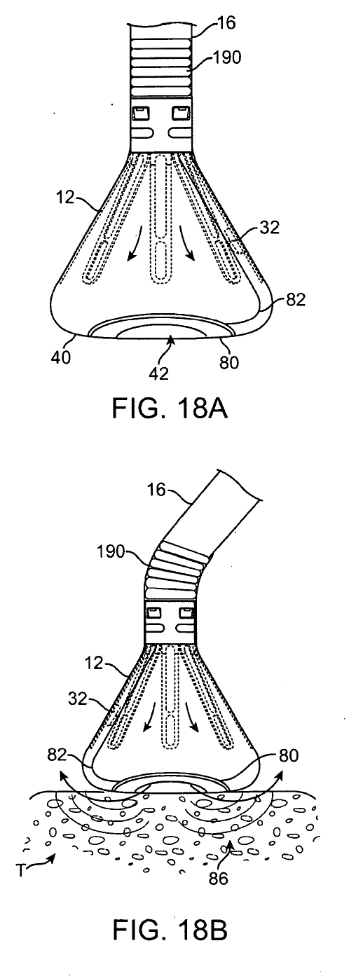

FIGS. 18A and 18B show side views of a variation where hood 12, having any of the electrode configurations described herein, may be coupled to deployment catheter 16 which has a flexible portion 190 along the catheter shaft proximal to hood 12. Flexible portion 190 may be comprised of a bendable segment which may be passively or actively curved. Such a structure allows hood 12 to conform on the tissue surface T regardless of the angle of approach which hood 12 takes relative to the tissue surface, as shown in FIG. 18B, even when hood 12 approaches the tissue surface at an angle less than 90 degrees. The flexible segment 190 may be covered by a securely fitted boot or covering to prevent the formation of blood clots due to the uneven shape of segment 190.

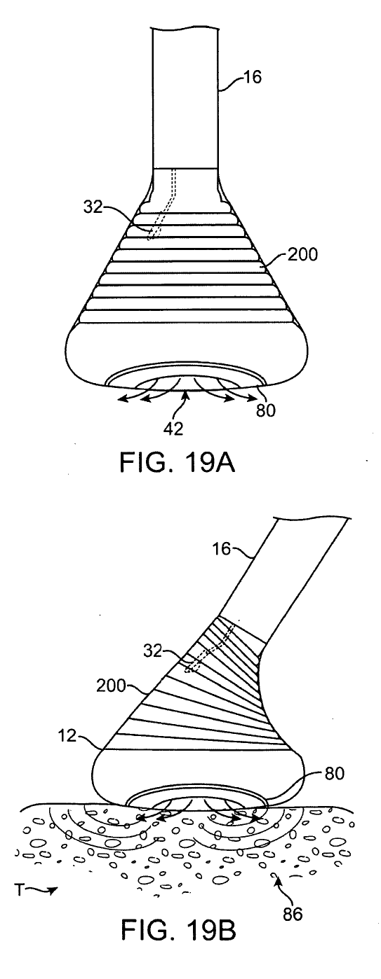

FIGS. 19A and 19B show another variation where the hood 12 may itself comprise a flexible portion 200. The hood 12 may include any of the electrode configurations described herein, such as electrode 80. As above, such a flexible, corrugating shape of hood 12 may facilitate engagement between hood 12 and the tissue by simply pushing the catheter 16 onto the tissue region to be inspected. FIG. 19B shows the configuration where the distal face of the hood 12 conforms to the tissue surface to enhance the effective visualization and ablation process regardless of the angle of approach between the hood 12 and the tissue surface.

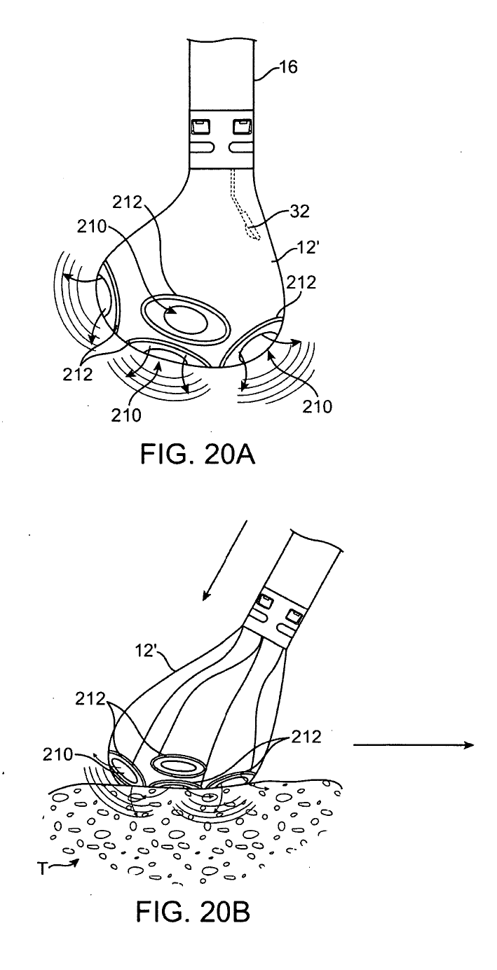

FIGS. 20A and 20B show side views of yet another variation where hood 12′ may comprise a flexible hood defining multiple apertures 210 over the hood surface. A select number or all apertures 210 may each have an electrode 212, such as a ring, disc-shaped, or any of the other electrode variations described herein, enclosing the apertures 210. The multiple electrodes 212 can also be used as sensors to collect electrical signals from the neighboring tissue regions and perform electrical signal mapping of the heart. The multiple electrodes 212 may be individually connected to individual respective energy delivery wires and depending on the desired region of the tissue to be ablated, selected electrodes 212 can be energized to deliver, e.g., RF energy. As shown in FIG. 20B, the floppy nature of the hood 12′ can be used to conform to undulating and/or trabeculated surfaces on the inner heart region and the hood can also be used to engage tissue at an angle instead of the hood 12′ being constrained at a perpendicular angle relative to the tissue region. Moreover, hood 12′ may be moved along the tissue region to ablate over a desired region of tissue. The saline purged through apertures 210 may not only clear the blood between the hood 12 and tissue surface but may also cool the underlying tissue during ablation.

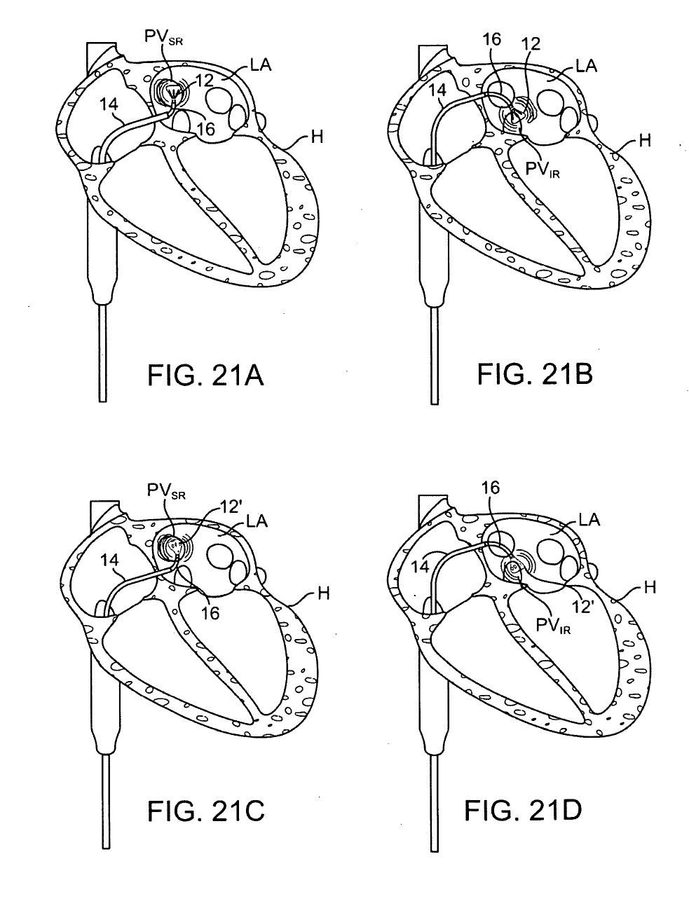

Due to direct full-color real-time visualization provided by hood 12 inside the heart H, the different regions of the transseptal walls can be easily recognized and selected for transseptal puncture to be performed. FIGS. 21A and 21B show the catheter advanced into the left atrium LA of the heart H through a transseptal puncture on the inferior transseptal wall. Accessing the left atrium LA from this position allows the deployment catheter 16 to be bent at a gentle bending radius, approximately 90 degrees, to access the superior right pulmonary vein PV_{SR} rather than having to incur a relatively tighter bending radius in a retroflexed manner for the hood 12 to access the same site perpendicularly. Likewise, the deployment catheter 16 could be inserted through the superior transseptal wall in order to perform ablation on the ostium of the inferior right pulmonary vein PV_{IR} as shown in FIG. 21B. Any of the hood and electrode variations, such as hood 12′, may be employed using the access paths described above, as shown in FIGS. 21C and 21D. Details of transseptal procedures and devices under direct visualization which may be utilized with apparatus and methods described herein are described in U.S. patent application Ser. No. 11/763,399 filed Jun. 14, 2007 (U.S. Pat. Pub. No. 2007/0293724 A1), which has been incorporated by reference herein above.

The applications of the disclosed invention discussed above are not limited to certain treatments or regions of the body, but may include any number of other treatments and areas of the body. Modification of the above-described methods and devices for carrying out the invention, and variations of aspects of the invention that are obvious to those of skill in the arts are intended to be within the scope of this disclosure. Moreover, various combinations of aspects between examples are also contemplated and are considered to be within the scope of this disclosure as well.

What is claimed is:

1. A tissue treatment system, comprising:
a reconfigurable hood which is capable of intravascular delivery in a low profile delivery configuration and expansion to a deployed configuration which defines an open area;
a fluid lumen in communication with the open area of the structure such that introduction of a conductive fluid through the lumen purges the open area of blood when the structure is further bounded by a tissue surface; and
an electrode supported by at least one support member, wherein the electrode is positionable adjacent to the open area in the deployed configuration and distally of the hood in the delivery configuration.

2. The system of claim 1 further comprising an imaging element within or along the hood such that the open area is contained within a visual field of the imaging element.

3. The system of claim 1 wherein the fluid lumen is positionable within or along the instrument.

4. The system of claim 1 wherein the electrode is positionable distal to an aperture defined in a membrane spanning the hood.

5. The system of claim 4 wherein the electrode defines a circular configuration approximating a size of the aperture.

6. The system of claim 4 wherein the hood further comprises a porous membrane positioned over the aperture, wherein the porous membrane further defines a plurality of openings.

7. The system of claim 4 wherein the membrane further comprises one or more ridges or barriers extending along the membrane.

8. The system of claim 1 wherein the electrode is supported by a first support member and a second support member, each member defining a curve or bend approximating a shape of the hood.

9. The system of claim 1 wherein the electrode is configured for provide ablation energy.

10. The system of claim 1 wherein the electrode is configured to sense or detect electrophysiological activity from the tissue surface.

11. A method of deploying a tissue treatment system, comprising:
intravascularly advancing an outer sheath to a tissue region of interest;
urging an electrode supported by at least one support member from the outer sheath;
urging a hood in a low profile delivery configuration from the outer sheath such that the hood is reconfigured into a deployed configuration and defines an open area, and wherein the electrode is positioned adjacent to the open area in the deployed configuration.

12. The method of claim 11 wherein intravascularly advancing comprises passing through an inferior or superior region of an atrial transseptal wall.

13. The method of claim 11 further comprising visualizing the tissue region bounded by the open area with an imager positioned within or along the hood.

14. The method of claim 11 further comprising purging blood from within the hood via a transparent fluid introduced through a fluid lumen in communication with the open area.

15. The method of claim 14 further comprising reducing a flow of the transparent fluid from the open area via a porous membrane defining a plurality of openings.

16. The method of claim 14 further comprising distributing the flow of transparent fluid between a membrane spanning the open area and the tissue region.

17. The method of claim 11 wherein the electrode is supported by a first support member and a second support member, each member defining a curve or bend approximating a shape of the hood.

18. The method of claim 11 further comprising ablating the tissue region of interest via the electrode in contact with the tissue region.

19. The method of claim 11 further comprising sensing or detecting electrophysiological activity from the tissue region via the electrode.

20. The method of claim 11 further comprising retracting the hood proximally into the outer sheath separately from the electrode.

21. A tissue treatment system, comprising:
an hood having a low profile intravascular delivery configuration and a deployed configuration, the hood in the deployed configuration defining an expanded interior volume having a distal open area;
an elongate body having a fluid lumen in communication with the volume such that introduction of a conductive fluid distally through the lumen purges the open area of blood when the deployed hood is disposed within a blood-filled site within a patient and the open area is adjacent a tissue surface;
an electrode; and
at least one support member extending distally from the elongate body so as to position the electrode adjacent to the open area in the deployed configuration and distally of the hood in the delivery configuration.
